(19) United States
(12) Patent Application Publication
(10) Pub. No.: US 2025/0082217 A1
(43) Pub. Date: Mar. 13, 2025

(54) APPARATUS AND METHOD OF PROCESSING BIOSIGNAL

(71) Applicant: SAMSUNG ELECTRONICS CO., LTD., Suwon-si (KR)

(72) Inventors: Soyoung LEE, Suwon-si (KR); Jinseok LEE, Yongin-si (KR); Seungseok LEE, Yongin-si (KR)

(73) Assignees: SAMSUNG ELECTRONICS CO., LTD., Suwon-si (KR); UNIVERSITY-INDUSTRY COOPERATION GROUP OF KYUNG HEE UNIVERSITY, Yongin-si (KR)

(21) Appl. No.: 18/884,994

(22) Filed: Sep. 13, 2024

(30) Foreign Application Priority Data
Sep. 13, 2023 (KR) 10-2023-0122081
Jan. 16, 2024 (KR) 10-2024-0006804

(51) Int. Cl. A61B 5/0245; A61B 5/00; A61B 5/308

(52) U.S. Cl. CPC A61B 5/0245; A61B 5/308; A61B 5/7221; A61B 5/7228; A61B 5/7253

(57) ABSTRACT

A biosignal processing method includes receiving a biosignal from a sensor, performing first signal processing based on variable frequency complex demodulation (VFCDM) on the biosignal, performing, once or more, second signal processing based on the VFCDM on a periodic signal having an instantaneous frequency as a center frequency, the instantaneous frequency being calculated based on the first signal processing, and estimating bio-information, based on at least one processing signal calculated based on the second signal processing performed once or more.

### CROSS-REFERENCE TO RELATED APPLICATIONS

This application claims priority to Korean Patent Application Nos. 10-2023-0122081, filed on Sep. 13, 2023, and 10-2024-0006804, filed on Jan. 16, 2024, in the Korean Intellectual Property Office, the disclosure of which are incorporated by reference herein in their entirety.

### BACKGROUND

The disclosure relates to an apparatus and method of processing a biosignal, and more particularly, to an apparatus and method of processing a biosignal through ultra-high resolution time-varying frequency analysis.

Biosignals such as an electrocardiogram (ECG) signal, a photoplethysmogram (PPG) signal, and an electromyography (EMG) signal have a temporal characteristic and a periodic characteristic, and thus, time and frequency analysis is needed for extracting a characteristic of a biosignal. Also, because noise penetrating into a biosignal has a temporal characteristic and/or a periodic characteristic, time and frequency analysis is needed for removing noise. Unstable information may be drawn in a case which separately analyzes a temporal characteristic and a periodic characteristic, and accordingly, it is needed to simultaneously analyze a time characteristic and a frequency characteristic. Moreover, because a biosignal has a nonlinear time-frequency characteristic, a signal processing method suitable for nonlinear time-frequency characteristic analysis is needed.

### SUMMARY

The disclosure provides a biosignal processing method and apparatus for ultra-high resolution time-varying frequency analysis of a biosignal, and moreover, provides a biosignal processing method and apparatus which may increase the accuracy of bio-information estimation through ultra-high resolution time-varying frequency analysis.

The disclosure is not limited to the aforesaid, but other objects not described herein will be clearly understood by those of ordinary skill in the art from descriptions below.

According to an aspect of the disclosure, a biosignal processing method may include: receiving a biosignal from a sensor; performing first signal processing on the biosignal, based on variable frequency complex demodulation (VFCDM); performing, at least once, second signal processing based on the VFCDM on a periodic signal having an instantaneous frequency as a center frequency, the instantaneous frequency being obtained based on the first signal processing; estimating bio-information, based on at least one processing signal obtained based on the second signal processing; and displaying the bio-information.

The second signal processing may be performed N times, where N is an integer of at least 2. (n+1)^{th} second signal processing may be performed on the periodic signal having a time-varying frequency as the center frequency, the time-varying frequency being obtained based on n^{th}-performed second signal processing, where n is an integer which is at least 1 and less than N.

The first signal processing may include: complex-demodulating the biosignal to generate a first signal; removing a signal component, which is greater than or equal to the center frequency, from the first signal to generate a second signal; reconstructing the biosignal, based on an amplitude and a phase, each obtained from the second signal; and calculating the instantaneous frequency from a reconstructed biosignal.

The second signal processing may include: complex-demodulating the periodic signal to generate a third signal; removing a signal component, which is greater than or equal to the center frequency, from the third signal to generate a fourth signal; reconstructing the periodic signal, based on an amplitude and a phase, each obtained from the fourth signal; and obtaining a time-varying frequency from a reconstructed periodic signal.

The obtaining of the time-varying frequency may include: performing Hilbert transform on the reconstructed periodic signal to obtain a time-varying amplitude and a time-varying phase of the reconstructed periodic signal; and calculating the time-varying frequency, based on the time-varying phase.

The second signal processing further may include normalizing the amplitude obtained from the fourth signal to obtain a weight, and the weight may be applied to the time-varying amplitude of the reconstructed periodic signal.

The estimating of the bio-information may include: obtaining the reconstructed periodic signal as a de-noised biosignal; and estimating a biological parameter, based on the de-noised biosignal.

The estimating of the bio-information may include: comparing the reconstructed periodic signal with the periodic signal; and determining quality of the biosignal, based on a comparison result.

The biosignal may include at least one of an electrocardiogram (ECG) signal and a photoplethysmogram (PPG) signal, and the estimating of the bio-information may include estimating a heart rate, based on a time-varying frequency obtained as a result of the second signal processing.

The biosignal may have periodicity based on time.

According to an aspect of the disclosure, a biosignal processing method may include: performing first signal processing based on variable frequency complex demodulation (VFCDM) on a first periodic signal obtained from a biosignal; performing second signal processing based on the VFCDM on a second periodic signal having an instantaneous frequency as a second center frequency, the instantaneous frequency being obtained based on the first signal processing; performing third signal processing based on the VFCDM on a third periodic signal having a time-varying frequency as a third center frequency, the time-varying frequency being obtained based on the second signal processing; estimating bio-information, based on at least one processing signal obtained based on the first signal processing, the second signal processing, and the third signal processing; and displaying the bio-information.

Each of the second signal processing and the third signal processing may include: complex-demodulating a periodic signal that is input; low-pass filtering a complex-demodulated signal by using a low-pass filter having a cut-off frequency which is lower than a center frequency; reconstructing the periodic signal, based on an amplitude and a phase, each obtained from a filtered signal; and obtaining the time-varying frequency from a reconstructed periodic signal.

The obtaining of the time-varying frequency may include: performing Hilbert transform on the reconstructed periodic signal; and calculating a time-varying amplitude, a time-varying phase, and the time-varying frequency, based on an analytic signal generated through the Hilbert transform.

Each of the second signal processing and the third signal processing may include normalizing the amplitude obtained from the filtered signal to obtain a weight, and the weight may be applied to the time-varying amplitude of the reconstructed periodic signal.

The estimating of the bio-information may include estimating the bio-information, based on the reconstructed periodic signal.

The biosignal may include at least one of an electrocardiogram (ECG) signal and a photoplethysmogram (PPG) signal, and the estimating of the bio-information may include estimating a heart rate, based on the time-varying frequency obtained as a result of the third signal processing.

A biosignal processing apparatus for processing a biosignal, may include: a display; a memory storing instructions; and at least one processor operatively connected to the display and the memory, the at least one processor being configured to execute the instructions to: perform first signal processing based on variable frequency complex demodulation (VFCDM) on a first periodic signal obtained from the biosignal; perform, at least once, second signal processing based on the VFCDM on a second periodic signal having an instantaneous frequency as a center frequency, the instantaneous frequency being obtained based on the first signal processing; estimate bio-information, based on at least one processing signal obtained based on the second signal processing; and instruct the display to display the bio-information.

The at least one processor may be further configured to execute the instructions to: complex-demodulate the second periodic signal; low-pass filter a complex-demodulated signal, based on a cut-off frequency which is lower than the center frequency; reconstruct the second periodic signal, based on an amplitude and a phase each obtained from a filtered signal; and obtain a time-varying frequency from a reconstructed second periodic signal.

The at least one processor may be further configured to execute the instructions to: normalize the amplitude obtained from the filtered signal to obtain a weight; and apply the weight to an amplitude obtained through Hilbert transform from the reconstructed second periodic signal.

The at least one processor may be further configured to execute the instructions to estimate the bio-information, based on the reconstructed second periodic signal.

The at least one processor may be further configured to execute the instructions to: compare the reconstructed second periodic signal with the second periodic signal; and determine quality of the biosignal, based on a comparison result.

The at least one processor may be further configured to execute the instructions to estimate a heart rate, based on a result of the second signal processing.

The at least one processor may be further configured to execute the instructions to estimate a heart rate, based on a time-varying spectrum obtained as a result of the second signal processing.

The biosignal processing apparatus further may include at least one sensor configured to measure the biosignal.

The at least one sensor may be further configured to provide at least one of an electrocardiogram (ECG) signal, a photoplethysmogram (PPG) signal, and an electromyography (EMG) signal to the at least one processor.

### BRIEF DESCRIPTION OF THE DRAWINGS

Embodiments will be more clearly understood from the following detailed description taken in conjunction with the accompanying drawings in which:

FIG. 1 is a block diagram illustrating an electronic device according to one or more embodiments;

FIG. 2A illustrates an example where an electronic device according to one or more embodiments is implemented as a wearable device;

FIG. 2B illustrates an example where an electronic device according to one or more embodiments is implemented as a wearable device;

FIG. 3A illustrates biosignals according to one or more embodiments;

FIG. 3B illustrates a biosignal to which noise is added according to one or more embodiments;

FIG. 4A illustrates a time-domain parameter of a biosignal according to one or more embodiments;

FIG. 4B illustrates a frequency-domain parameter of a biosignal according to one or more embodiments;

FIG. 5 is a flowchart illustrating a signal processing method according to one or more embodiments;

FIG. 6 illustrates in detail signal processing based on iterative time-varying frequency complex demodulation (iTFCDM) according to one or more embodiments;

FIG. 7 illustrates signal processing based on iTFCDM according to one or more embodiments;

FIG. 8A illustrates a time-frequency spectrum of time-series signals according to one or more embodiments;

FIG. 8B illustrates a time-frequency spectrum of time-series signals according to one or more embodiments;

FIG. 8C illustrates a time-frequency spectrum of time-series signals according to one or more embodiments;

FIG. 8D illustrates a time-frequency spectrum of time-series signals according to one or more embodiments;

FIG. 9 is a flowchart illustrating a signal processing method according to one or more embodiments;

FIG. 10A illustrates a time-frequency spectrum of a photoplethysmogram (PPG) signal according to one or more embodiments;

FIG. 10B illustrates a time-frequency spectrum of a photoplethysmogram (PPG) signal according to one or more embodiments;

FIG. 11 is a flowchart illustrating a signal processing method according to one or more embodiments;

FIG. 12A illustrates an electrocardiogram (ECG) signal according to one or more embodiments;

FIG. 12B illustrates a reconstructed ECG signal generated based on signal processing according to one or more embodiments;

FIG. 13 is a flowchart illustrating a signal processing method according to one or more embodiments;

FIG. 14A illustrates a reconstructed ECG signal generated based on signal processing according to one or more embodiments;

FIG. 14B illustrates a reconstructed ECG signal generated based on signal processing according to one or more embodiments;

FIG. 15A illustrates a biosignal monitoring system according to one or more embodiments; and

FIG. 15B illustrates a biosignal monitoring system according to one or more embodiments.

### DETAILED DESCRIPTION OF THE EMBODIMENTS

Hereinafter, embodiments will be described in detail with reference to the accompanying drawings.

Elements described as “module” or “part” or “unit” or “device” may be physically implemented by analog and/or digital circuits including one or more of a logic gate, an integrated circuit, a microprocessor, a microcontroller, a memory circuit, a passive electronic component, an active electronic component, and the like.

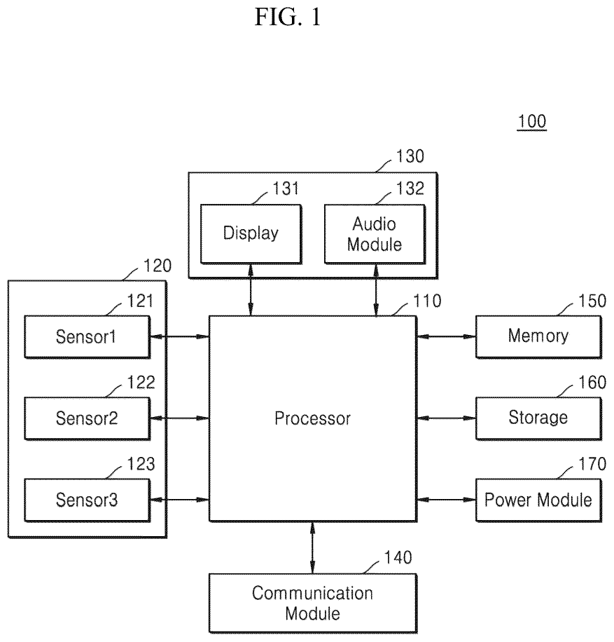

FIG. 1 is a block diagram illustrating an electronic device 100 according to one or more embodiments.

Referring to FIG. 1, the electronic device 100 may include a processor 110, a sensor module 120, an input/output (I/O) device 130, a communication module 140, a memory 150, a storage 160, and a power module 170. The electronic device 100 is not limited thereto and may further include various elements.

The electronic device 100 may be a user-wearable device for monitoring a biosignal of a user. The user may wear the electronic device 100 on a body part such as an arm, a leg, or a neck, and the electronic device 100 may sense biosignals of the user by using a sensor (for example, first to third sensors 121 to 123) included in the sensor module 120 and may monitor a health status of the user.

The sensor module 120 may include a plurality of sensors (for example, the first sensor 121, the second sensor 122, and the third sensor 123). In FIG. 1, for example, three sensors are illustrated, but the disclosure is not limited thereto and the sensor module 120 may include two sensors or four or more sensors.

The plurality of sensors may include biosignal measurement sensors which measure the biosignal of the user. The plurality of sensors may sense different biosignals. In one or more embodiments, at least one of the plurality of sensors may be a motion sensor which senses a motion of the user. In one or more embodiments, at least one of the plurality of sensors may be a voice sensor (for example, a microphone) which senses a voice of the user.

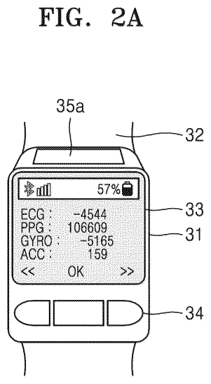

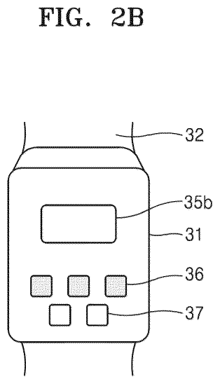

For example, the first sensor 121 may be an electrocardiogram (ECG) sensor. The ECG sensor may measure ECG of the user to generate an ECG signal. The ECG sensor may include a plurality of electrodes (or a plurality of pads) (for example, 35a of FIGS. 2A and 35b of FIG. 2B) and an analog front end (AFE).

An electrical signal may be applied to a skin of the user through at least one of a plurality of electrodes, and an ECG signal representing electrical activity appearing in heart may be output through at least another electrode of the plurality of electrodes, based on the electrical signal.

In one or more embodiments, as illustrated in FIG. 2A, at least one of the plurality of electrodes may be disposed to contact the skin of the user, and at least another electrode of the plurality of electrodes may contact the skin of the user when a body part (for example, a finger) of the user contacts a corresponding electrode intentionally. Therefore, the first sensor 121 may measure ECG in response to a request (for example, contact) of the user, or may measure ECG in response to a request of the processor 110. The AFE may amplify and analog-digital convert a measured electrical signal to generate an ECG signal.

The first sensor 121 may transfer the ECG signal to the processor 110. For example, the transfer of the ECG signal may be performed based on an interface based on a serial peripheral interface (SPI). However, the disclosure is not limited thereto, and one or more interface protocols of high speed serial interface (HSSI) protocols such as inter-integrated circuit (I2C), improved inter integrated circuit (I3C), mobile industry processor interface (MIPI), universal asynchronous receiver/transmitter (UART), embedded display port (eDP), low-voltage differential signaling (LVDS), universal serial interface (USI), ultra path interface (UPI), and enhanced reduced voltage differential signal transmission (eRVDS) may be applied between the first sensor 121 and the processor 110. However, the disclosure is not limited thereto, and one interface protocol of various HSSI protocols described above may be applied to the first sensor 121 and the processor 110.

For example, the second sensor 122 may be a photoplethysmogram (PPG) sensor. The PPG sensor may measure a pulse wave of the user to generate a PPG signal. The PPG sensor may measure the pulse wave of the user, based on principle that the amount of absorbed light is changed by heartbeat when light is irradiated onto the skin of the user.

The second sensor 122 may include a light-emitting module and a light receiving module, the light-emitting module may include one or more light-emitting devices (for example, a light-emitting diode (LED)), and the light receiving module may include one or more light receiving devices (for example, a photo sensor including a photodetector). In one or more embodiments, the second sensor 122 may further include an AFE, and the AFE may drive the light-emitting module in response to a measurement request from the processor 110 and may analog-digital convert a signal from the light receiving module to transfer to the processor 110.

The second sensor 122 may measure the pulse wave of the user to generate the PPG signal. The light-emitting module and the light receiving module of the second sensor 122 may contact the skin of the user, and thus, may always measure the pulse wave of the user. For example, the second sensor 122 may continuously measure a pulse wave in a state where there is no perception of the user.

The second sensor 122 may transfer the PPG signal to the processor 110. For example, the transfer of the PPG signal may be performed based on an interface based on SPI. However, the disclosure is not limited thereto, and one interface protocol of various HSSI protocols described above may be applied to the second sensor 122 and the processor 110.

The third sensor 123 may be a motion sensor. For example, the third sensor 123 may be implemented as an inertial measurement unit (IMU). The IMU may include an acceleration sensor and a gyroscope. The IMU may further include a geomagnetic sensor. The third sensor 123 may measure a motion of the user (hereinafter referred to as a motion) (for example, posture change, a change speed of position movement, and the amount of displacement) to generate an IMU signal (for example, a three-axis accelerometer signal). The IMU signal may be referred to as a motion sensing signal. The third sensor 123 may continuously measure a motion of the user to generate the IMU signal.

The third sensor 123 may transfer the IMU signal to the processor 110. For example, the transfer of the IMU signal may be performed based on an interface based on I2C. However, the disclosure is not limited thereto, and one interface protocol of various HSSI protocols described above may be applied to the third sensor 123 and the processor 110.

In one or more embodiments, the sensor module 120 may further include another biosensor. For example, the sensor module 120 may further include a sensor which measures a bio-impedance of the user, or a sensor which senses a variation or a status of sweat, blood, urine, or iris. For example, the sensor module 120 may further include a respiratory (RSP) senor, a galvanic skin response (GSR) senor, EDA (electrodermal activity) senor, Electromyography (EMG) senor, BCG (ballistocardiogram: BCG) senor, a sweat sensor for sensing hydration or dehydration, an iris sensor, an electroretinogram (ERG) sensor, an electrooculography (EOG) sensor, a body temperature sensor, or an electroencephalogram (EEG) sensor. In one or more embodiments, the sensor module 120 may further include a sensor such as a voice sensor in addition to a biosensor.

The processor 110 may control an overall operation of the electronic device 100 and may control the elements (for example, the sensor module 120, the I/O module 130, the communication circuit 140, the memory 150, the storage 160, and the power module 170). In one or more embodiments, the processor 110 may include a micro control unit (MCU). However, the disclosure is not limited thereto, and the processor 110 may include a processor such as a central processing unit (CPU) or a micro processing unit (MPU).

The processor 110 may signal-process the biosignal (for example, the PPG signal, the ECG signal, and the IMU signal) received from the sensor module 120 and may monitor a health status of the user, based on a signal processing result.

The processor 110 may perform ultra-high resolution time-varying frequency analysis on the biosignal through signal processing based on an iterative time-varying frequency complex demodulation (iTFCDM) scheme (algorithm) described below on the biosignal. Here, iTFCDM may be a method which iteratively performs signal processing based on variable frequency complex demodulation (VFCDM) to amplify a signal and reduce noise, based on an increase in the number of iterations.

VFCDM may be a nonlinear time-frequency characteristic analysis method based on Hilbert transform. The processor 110 may iteratively perform signal processing based on VFCDM to continuously update a reconstructed biosignal generated based on VFCDM.

In one or more embodiments, the processor 110 may normalize an amplitude of the reconstructed biosignal and may apply a normalized value to an instantaneous amplitude which is dependent on a time and a frequency and is calculated based on an analytic signal drawn by Hilbert transform. Accordingly, a signal of a center frequency may be enhanced.

A biosignal may have a temporal characteristic and a periodic characteristic. Noise may also have a temporal characteristic and/or a periodic characteristic. Therefore, time and frequency analysis may be needed for extracting a characteristic of a biosignal. Here, in a case where the analysis of a temporal characteristic and a periodic characteristic (i.e., a frequency characteristic) is separately performed, information may not be normally analyzed because being unstable. Therefore, time analysis and frequency analysis may be simultaneously needed.

In the electronic device 100 according to one or more embodiments, the processor 110 may perform ultra-high resolution time-varying frequency analysis on a biosignal (e.g., a periodic signal obtained from the biosignal) through signal processing based on iTFCDM and may estimate bio-information, based on an analysis result. Accordingly, the electronic device 100 may provide accurate bio-information to the user. The processor 110 may estimate bio-information, based on at least one processing signal calculated through signal processing based on iTFCDM.

In one or more embodiments, a biosignal may include an ECG signal (or a PPG signal), and the processor 110 may estimate, as a heart rate, a time-varying frequency generated through signal processing based on iTFCDM on the ECG signal. Alternatively, the processor 110 may estimate a heart rate, based on a time-frequency spectrum generated through signal processing based on iTFCDM.

In one or more embodiments, the processor 110 may obtain, as a de-noised biosignal, a reconstructed biosignal (e.g., a reconstructed periodic signal) generated in a signal processing process based on iTFCDM and may estimate a biometric parameter (for example, a heart rate, a breathing rate, oxygen saturation, blood pressure, heart rate variability, stress, and sleep apnea), based on the de-noised biosignal.

In one or more embodiments, the processor 110 may compare a biosignal (an original biosignal) with the reconstructed biosignal generated in the signal processing process based on iTFCDM and may determine the quality of the biosignal, based on a comparison result.

In one or more embodiments, the processor 110 may enhance the biosignal through signal processing based on iTFCDM. For example, the processor 110 may increase a signal-to-noise ratio (SNR) of the biosignal. The electronic device 100 may output an enhanced biosignal. Also, the processor 110 may perform signal processing based on iTFCDM in a signal extraction process for increasing an SNR of a sensing signal such as a voice signal in addition to a biosignal.

The processor 110 may output estimated bio-information through a display 131 and/or an audio module 132. Also, the processor 110 may transmit the estimated bio-information to an external device (for example, a medical server) through the communication module 140. The processor 110 may output an enhanced voice signal through the audio module 132, or may transmit the enhanced voice signal to an external device through the communication module 140.

The display 131 may display various information, based on control by the processor 110. For example, the display 131 may display bio-information about the user such as a heart rate and oxygen saturation. The display 131 may display atrial fibrillation detection information (whether atrial fibrillation occurs or not), arrhythmia information (the presence of arrhythmia and/or the kind of arrhythmia), or suspected disease information. The display 131 may display information requesting a motion of a user, and for example, may display information which issues a request to contact a finger with an ECG measurement electrode for ECG measurement and recommendation information for visiting a hospital.

The display 131 may be configured as at least one of various displays such as a liquid crystal display (LCD), a thin film transistor LCD (TFT-LCD), an organic light-emitting diode (OLED), a light-emitting diode (LED), an active matrix organic LED (AMOLED), a micro LED, a flexible display, and a three-dimensional (3D) display. In one or more embodiments, the display 131 may be implemented as a type of touch screen. In one or more embodiments, the display 131 may be implemented as an extended display or a flexible display.

The audio module 132 may output a sound, and for example, the audio module 132 may include at least one of an audio codec, a microphone (MIC), a receiver, an earphone output, and a speaker. The audio module 132 may output, as an audio signal, information associated with a body status of a user, information associated with symptom of a health status of the user, or additional information. For example, when atrial fibrillation is detected, the audio module 132 may output an alarm representing that the atrial fibrillation occurs. In one or more embodiments, the audio module 132 may receive a voice signal of the user or an external voice signal through a microphone and may output an enhanced voice signal through a speaker, based on iTFCDM.

The communication module 140 may communicate with an external device. In one or more embodiments, the communication module 140 may include a Bluetooth module. However, the disclosure is not limited thereto, and for example, the communication module 140 may include a communication interface accessible to wireless local area network (WLAN), such as wireless fidelity (Wi-Fi), wireless personal area network (WPAN), wireless universal serial bus (USB), Zigbee, near field communication (NFC), or radio frequency identification (RFID), or mobile cellular network such as 3rd generation (3G), 4th generation (4G), or long term evolution (LTE). In one or more embodiments, the communication module 140 may further include a communication interface accessible to a wired local area network.

The communication module 140 may transmit information associated with a body status of a user, information associated with symptom of a health status of the user, or additional information to an external electronic device (for example, a smartphone of the user). The communication module 140 may transmit a measured biosignal (for example, ECG data) to the external electronic device. The external electronic device may transmit the biosignal to the medical server. In one or more embodiments, the communication module 140 may access a network (for example, an access point or a mobile network) to directly transmit the ECG data to the medical server.

The memory 150 may be implemented as a volatile memory, such as dynamic random access memory (DRAM) or static random access memory (SRAM), or a non-volatile resistive memory such as phase-change random access memory (PRAM) or resistive random access memory (ReRAM).

An operating program or an application program executed in the processor 110 may be loaded into the memory 150 and executed by the processor 110. A program, including instructions (or codes) for implementing a function of the at least one processor 110 described above (for example, a function of estimating bio-information based on a signal processing result and/or signal processing based on iTFCDM or at least one processing signal generated in a signal processing process), may be loaded into the memory 150 and executed by the (at least one) processor 110.

Also, the memory 150 may store data which is to be processed in the processor 110 or data generated by the processor 110. For example, the memory 150 may temporarily store a biosignal measurement record (for example, the number of measurements, a measurement time, etc.), estimated bio-information, and suspected disease information.

In FIG. 1, the memory 150 is illustrated as a separate element provided independently from the processor 110, but is not limited thereto and in one or more embodiments, the memory 150 may be integrated into the processor 110. The memory 150 may be implemented as a storage region in the processor 110.

The storage 160 may be implemented as a non-volatile memory device such as NAND flash memory or a resistive memory, and for example, the storage 160 may be provided as a memory card (for example, multimedia card (MMC), embedded multi-media card (eMMC), secure digital (SD) card, or micro SD). The storage 160 may store data generated by the processor 110. The storage 160 may store a biosignal measurement record (for example, the number of measurements, a measurement time, etc.), bio-information about the user, and suspected disease information. Also, the storage 160 may store the measured biosignal.

The power module 170 may include a battery, a charging circuit, and a power management unit (PMU). In one or more embodiments, the PMU may be integrated into the processor 110. The power module 170 may generate and provide voltages used in the electronic device 100, based on power supplied from the battery or an external power source. The power module 170 may also charge the battery, based on external power. The PMU may manage power of elements of a battery of each element. For example, the PMU may supply power to each element and may adjust an operation frequency or a level (for example, a voltage level) of power supplied to each element, based on an operating state of the electronic device 100 or an operating state of each element. Also, the PMU may cut off power.

FIGS. 2A and 2B illustrate an example where an electronic device 100 according to one or more embodiments is implemented as a wearable device. FIG. 2A may illustrate a first surface (for example, a front-side surface) of the electronic device 100, and FIG. 2B may illustrate a second surface (for example, a backside surface) of the electronic device 100.

Referring to FIGS. 2A and 2B, the electronic device 100 may be, for example, a watch-type wearable device capable of being worn on a wrist of a user or a wearable device capable of being worn on another part (for example, a head, a forearm, a thigh, or another part of a human body capable of ECG measurement) of a human body.

A display 33, an input button 34, a plurality of electrodes (for example, a first electrode 35a and a second electrode 35b), at least one light-emitting device 36, and at least one light receiving device 37 may be disposed in a housing 31 configuring an external appearance of the electronic device 100. An assistant strap 32 enabling the electronic device 100 to be worn on a body of the user may be connected to the housing 31. Some elements (for example, the processor 110, the communication module 140, at least one sensor (for example, the third sensor 123), the audio module 132, the memory 150, the storage 160, and the power module 170) described above with reference to FIG. 1 may be provided in the housing 31.

The first electrode 35a and the second electrode 35b may be electrodes for detecting the same biosignal (for example, an ECG signal). The first electrode 35a may be disposed on the first surface of the electronic device 100, and the second electrode 35b may be disposed on the second surface of the electronic device 100. The at least one light-emitting device 36 and the at least one light receiving device 37 may be disposed on the second surface of the electronic device 100. The at least one light-emitting device 36 and the at least one light receiving device 37 may be devices for detecting a PPG of the user and may be included in a second sensor (122 of FIG. 1).

In one or more embodiments, elements for detecting another biosignal (for example, breathing, EDA, EMG, or EOG) may be further provided in the first surface or the second surface of the electronic device 100.

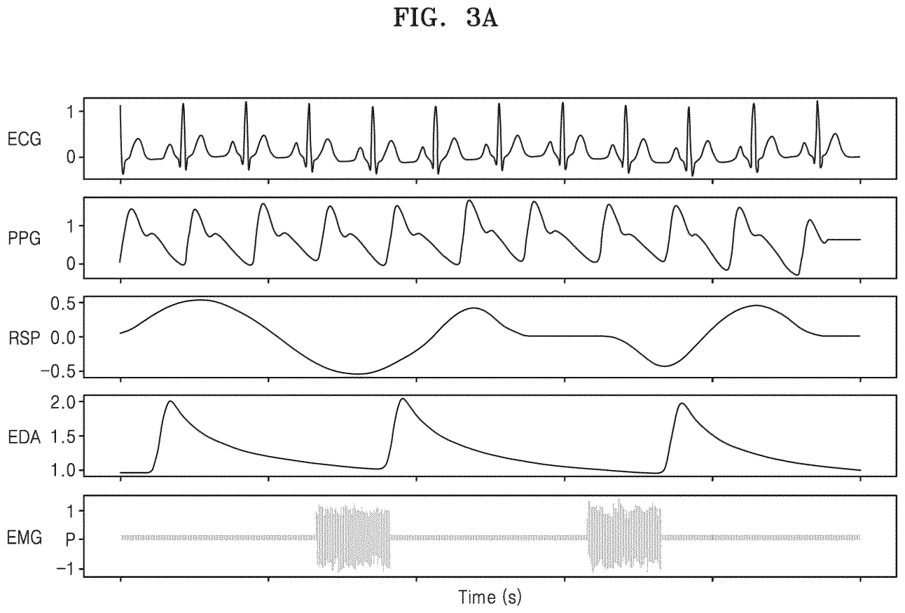

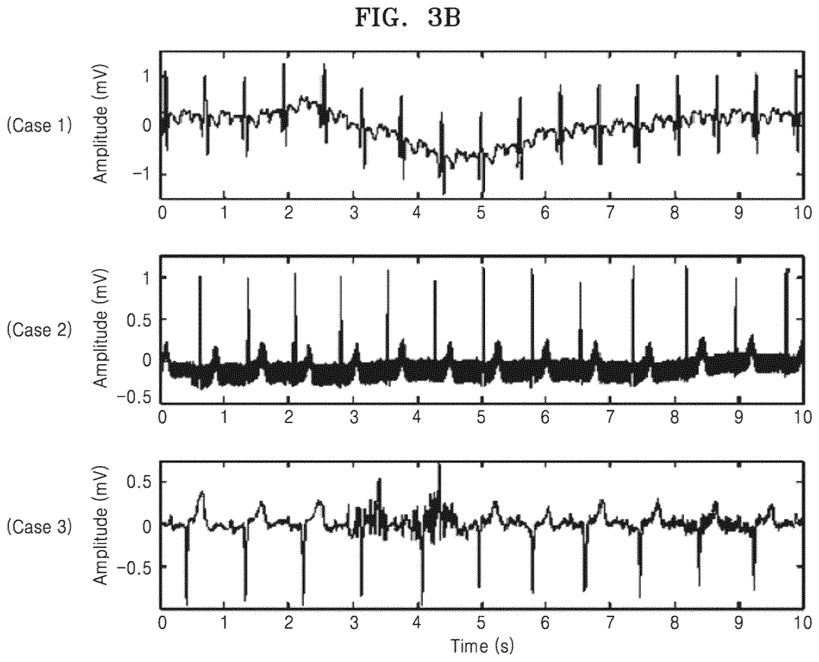

FIG. 3A illustrates biosignals, and FIG. 3B illustrates a biosignal to which noise is added. In graphs of FIGS. 3A and 3B, the abscissa axis represents a time, and the ordinate axis represents an amplitude of a biosignal.

Referring to FIG. 3A, the biosignal may include signals such as an ECG signal, a PPG signal, an RSP signal, an EDA signal, and an EMG signal.

The ECG signal may be a biosignal representing the EDA signal occurring in a heartbeat process. The ECG signal may include five waveforms P, Q, R, S, and T where heights and intervals differ, and P, Q, R, S, and T waves may be iteratively generated. QRS complex may be a considerable feature of the ECG signal and may represent a heartbeat status.

The PPG signal may be a biosignal representing a change in blood flow rate with respect to the diastole and systole of a heart. The PPG signal may be generated by measuring an electrical signal, obtained from light reflected by a change in bloodstream, in skin. The PPG signal may be used for representing a periodicity of a signal waveform and may include quasi-period pulses including peaks and valleys enabling the measurement of a heart rate.

The RSP signal may be a biosignal used for estimating a breathing rate of the user. The RSP signal may include a Sin or Cos waveform.

The EDA signal may represent an electrical conductance of the skin of the user and may be measured based on the amount of current flowing through a plurality of electrodes attached on the skin of the user. The EDA signal may include a skin conductance level (SCL) which is a low frequency component and a skin conductance response (SCR) which is a high frequency component, and a sympathetic skin response may be estimated based on a change in EDA signal.

The EMG signal may be an electrical signal which is generated when muscle contracts and relaxes and may represent a frequency characteristic in a wide frequency band (for example, about 10 Hz to about 3 KHz).

As described above, each biosignal may be a periodic signal where a pattern of a certain signal is periodically iterated. Furthermore, a period of a biosignal may vary over time, and thus, the biosignal may have a temporal characteristic and a periodic characteristic. Therefore, time and frequency analysis may be needed for extracting a characteristic of a biosignal.

FIG. 3B illustrates noise types of an ECG signal. The ECG signal may be a micro biosignal where a maximum magnitude of a waveform is millivolt (mV) in unit. Various kinds of noises may be in the ECG signal when measuring ECG, and the noises may respectively have unique characteristics.

Case1 may represent a baseline wander of the ECG signal. The baseline wander may be a criterion for measuring a feature point of the ECG signal. A low frequency artifact occurring due to breath, an electrically charged electrode, or a motion of a user may cause the baseline wander of the ECG signal, and accurate diagnosis may not be performed when an artifact factor is not removed, in the ECG signal where there is the baseline wander.

Case2 may represent noise caused by power line interference. A measurement environment of the ECG signal may be mostly exposed to power line interference of about 50 Hz to about 60 Hz. Power line interference may cause the distortion of a certain period in ECG signal analysis. A period, which is a significant factor in arrhythmia diagnosis like P wave and R wave, may have a small amplitude and interval similar to a component of power line interference, and thus, when the ECG signal is distorted, an error of a diagnosis result may occur due to inaccurate detection.

Case3 may represent noise caused by the EMG signal. EMG noise may a frequency characteristic in a wide band, and impulsive noise may have a characteristic where the impulsive noise occurs in a needle shape in a very short interval of several ms (millisecond).

As described above, noise may also have a temporal characteristic and/or a periodic characteristic, and a temporal characteristic and/or a periodic characteristic may differ based on the type of noise. Accordingly, time and frequency analysis may be needed for removing noise from a biosignal.

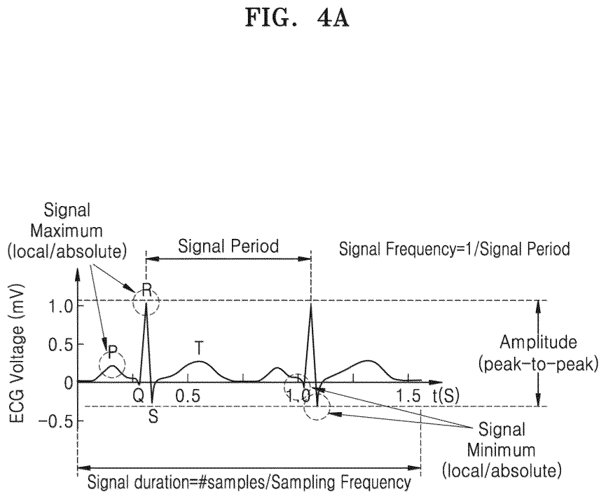

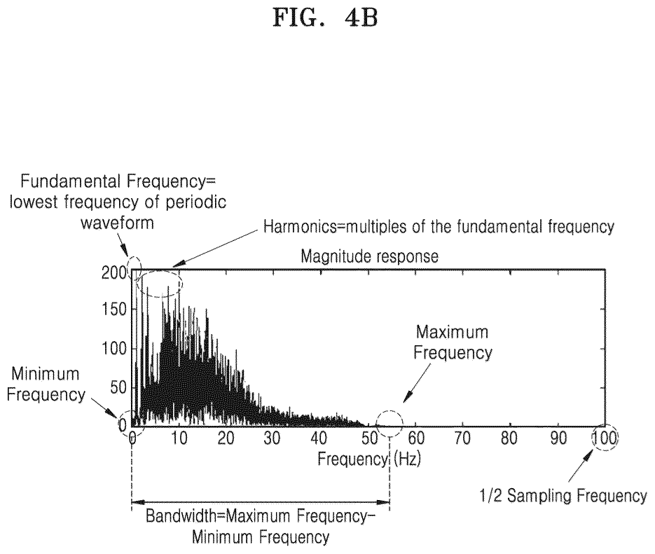

FIGS. 4A and 4B illustrate a time-domain parameter and a frequency-domain parameter of an ECG signal (ECG voltage), respectively. The time-domain parameter may represent a temporal characteristic, and the frequency-domain parameter may represent a frequency characteristic.

Referring to FIG. 4A, a parameter of a time domain may include a signal duration, a signal period, a signal frequency, a signal maximum value and a signal minimum value, and an amplitude.

The signal duration may be a value obtained by dividing the number of samplings by a sampling frequency. The ECG signal may include P, Q, R, S, and T waves which are iteratively generated. An interval between R peak and adjacent R peak may be a period of a signal, and an inverse number value of the period of the signal may be a frequency of the signal. R peak (peak of R wave) may be a maximum value of a signal, and S peak (peak of S wave) may be a minimum value of the signal. A voltage difference between R peak and S peak in one period may be an amplitude of the signal.

Referring to FIG. 4B, a parameter of a frequency domain may include a fundamental frequency, a minimum frequency, a maximum frequency, harmonics, and a bandwidth. The fundamental frequency may be a lowest frequency of a periodic waveform, and the harmonics may be a multiple of the fundamental frequency. The bandwidth may be a difference between the maximum frequency and the minimum frequency.

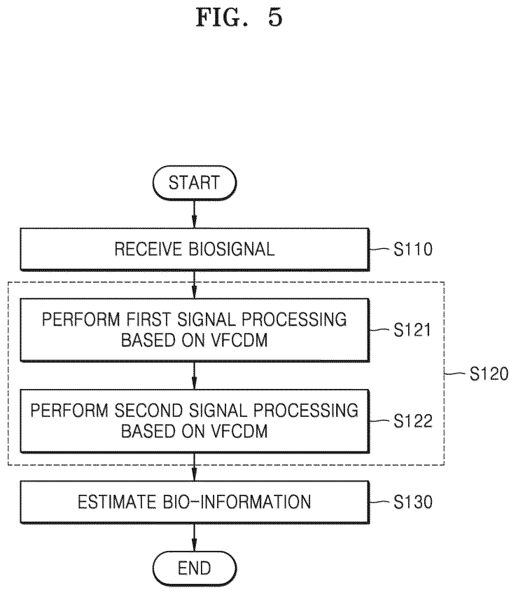

FIG. 5 is a flowchart illustrating a signal processing method according to one or more embodiments. FIG. 5 illustrates signal processing based on iTFCDM and may be performed by at least one processor (for example, the processor 110 of FIG. 1). Therefore, the description of the signal processing by the processor 110 described above with reference to FIG. 1 may be applied to the present embodiment.

Referring to FIG. 5, the processor may receive a biosignal from a sensor in operation S110. As described above with reference to FIG. 3A, the biosignal may be a periodic signal.

The processor may perform signal processing based on iTFCDM on a biosignal (for example, a first periodic signal) in operation S120. The signal processing based on iTFCDM may include first signal processing S121 and second signal processing S122 based on VFCDM.

The processor may perform the first signal processing based on VFCDM on the biosignal in operation S121. The periodic signal may be represented by a frequency, an amplitude, and a phase. The biosignal may include a center frequency, an instantaneous amplitude, an instantaneous phase, and direct current (DC) power. In a firs signal processing operation, a reconstructed biosignal from which noise has been removed may be calculated through complex demodulation and low-pass filtering, and an instantaneous frequency of the reconstructed biosignal may be calculated through Hilbert transform on the reconstructed biosignal.

The processor may perform the second signal processing based on VFCDM on the second periodic signal having an instantaneous frequency as a center frequency in operation S122.

The second periodic signal may include an instantaneous frequency, an instantaneous amplitude, an instantaneous phase, and DC power each calculated in first signal processing operation S121. In a second signal processing operation on the second periodic signal, a reconstructed second periodic signal from which noise has been removed may be calculated through complex demodulation and low-pass filtering, and a time-varying frequency of the reconstructed second periodic signal may be calculated through Hilbert transform on the reconstructed second periodic signal. Also, a time-varying amplitude and a time-varying phase of the reconstructed second periodic signal may be calculated.

Here, the second signal processing may be performed once or more. The processor may perform the second signal processing once or iteratively. As the second signal processing is iteratively performed, the reconstructed second periodic signal may be updated. A signal component of the reconstructed second periodic signal may be amplified, and noise may be reduced.

The processor may estimate bio-information, based on at least one processing signal calculated through the second signal processing which is performed once or more. For example, the processor may estimate bio-information, based on reconstructed signals (for example, the reconstructed biosignal or the reconstructed second periodic signal) calculated through the second signal processing which is performed once or more, or may estimate bio-information, based on a time-varying frequency, a time-varying amplitude, and/or a time-varying phase each output as a result of the second signal processing which is performed once or more.

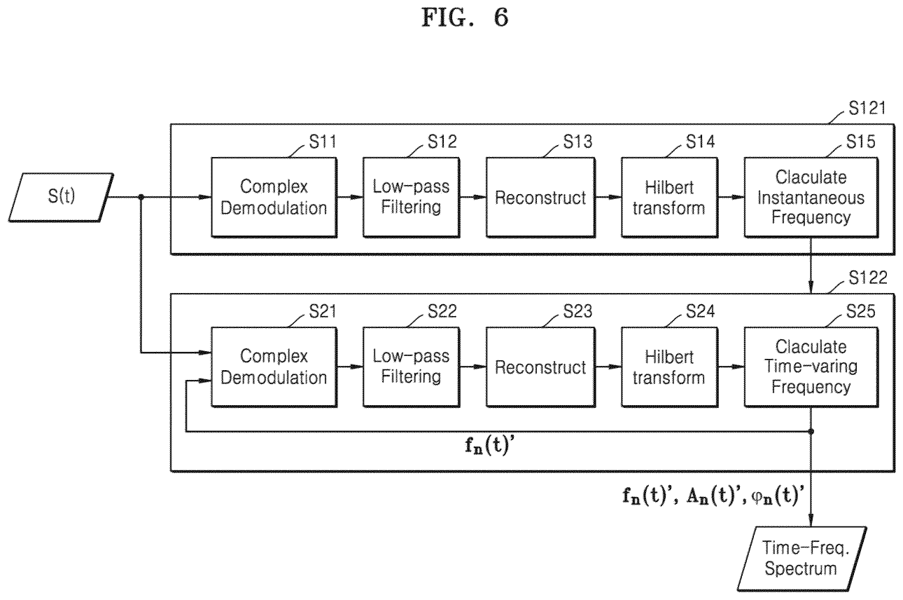

FIG. 6 illustrates in more detail signal processing based on iTFCDM according to one or more embodiments.

A biosignal may be represented by a first periodic signal S(t) of the following Equation 1.

$$S ( t ) = A ( t ) cos ( 2 π {f}_{c} t + φ ( t ) ) + C ( t ) \qquad [ Equation 1 ]$$

Here, C(t) may denote DC power, A(t) may denote an amplitude, f_{c} may denote a center frequency, and φ(t) may denote a phase. The center frequency f_{c} may be a fixed frequency. Here, when it is assumed that the center frequency of the biosignal is 0, the processor may shift the center frequency up to fs/2 by 0.5 units to detect the center frequency f_{c}. Here, fs may denote a sampling rate of the biosignal.

The processor may perform the first signal processing based on VFCDM on the first periodic signal S(t) in operation S121. The first signal processing S121 may include complex demodulation S11, low-pass filtering S12, signal reconstruction S13, Hilbert transform S14, and instantaneous frequency calculation S15.

The processor may complex-demodulate the first periodic signal S(t) in operation S11. The first periodic signal S(t) may be multiplied by

$${e}^{- j 2 π {f}_{c} t} ,$$

and a result thereof may be expressed as the following Equation 2. A center frequency of the first periodic signal S(t) may be shifted to 0. In other words, the center frequency of the first periodic signal S(t) may be modulated.

$${S}_{c} ( t ) = S ( t ) \cdot {e}^{- j 2 π {f}_{c} t} = \frac{A ( t )}{2} {e}^{j φ ( f )} + \frac{A ( t )}{2} {e}^{- j ( 4 π {f}_{c} t + φ ( t ) )} + C ( t ) {e}^{- j 2 π {f}_{c} t} \qquad [ Equation 2 ]$$

The processor may perform low-pass filtering on a complex-demodulated signal S_{c}(t) in operation S12. The processor may apply a low-pass filter, having a cut-off frequency which is less than the center frequency f_{c}, to a complex-demodulated signal S_{c}(t). Therefore, a second complex exponential term

$$\frac{A ( t )}{2} {e}^{- j ( 4 π {f}_{c} t + φ ( t ) )}$$

and a third exponential term

$$C ( t ) {e}^{- j 2 π {f}_{c} t}$$

may be removed in Equation 2, and a filtered signal Sₜₚ(t) of the following Equation 3 may be obtained. Accordingly, a signal component of the center frequency f_{c} or more may be removed.

$${S}_{l p} ( t ) = \frac{A ( t )}{2} {e}^{j φ ( t )} \qquad [ Equation 3 ]$$

An amplitude A(t) and a phase φ(t) may be respectively expressed as the following Equation 4 and Equation 5.

$$A ( t ) = 2 \vert {S}_{l p} ( t ) \vert \qquad [ Equation 4 ]$$

$$φ ( t ) = {tan}^{- 1} ( \frac{imag ( {S}_{l p} ( t ) )}{real ( {S}_{l p} ( t ) )} ) \qquad [ Equation 5 ]$$

Here, real(·) and imag(·) may respectively denote a real number part and an imaginary number part of a complex signal.

The processor may reconstruct the first periodic signal (i.e., the biosignal) by using the amplitude A(t) and the phase φ(t) in operation S14, and a reconstructed first periodic signal sᵣ(t) may be expressed as the following Equation 6.

$${s}_{r} ( t ) = A ( t ) cos ( 2 π {f}_{c} t + φ ( t ) ) \qquad [ Equation 6 ]$$

Comparing with the first periodic signal S(t), in the reconstructed first periodic signal Sᵣ(t), it may be seen that a signal (for example, a biosignal where a characteristic is to be extracted) is amplified and noise is removed.

The processor may apply Hilbert transform to the reconstructed first periodic signal sᵣ(t) so as to detect an instantaneous frequency and phase in operation S14. Hilbert transform may shift only a phase of a real number signal by ±90 degrees to enable an amplitude and a phase to be easily analyzed. An analytic signal of the following Equation 7 may be calculated by Hilbert transform.

$$Y ( t ) = {s}_{r} ( t ) + jH ( {s}_{r} ( t ) ) \qquad [ Equation 7 ]$$

The analytic signal Y(t) may obtain a real number part of the first periodic signal sᵣ(t) and may generate another function of an imaginary number variable H(sᵣ(t)). Here, H(·) may denote a Hilbert transform operator. H(·) may assign a phase variation of ±90 degrees (π/2 radians) to all frequency components, and a sign of a variation may be changed based on a sign of a frequency. A real number part of the analytic signal may represent an original signal, and an imaginary number part may represent an instantaneous phase of a signal.

The processor may calculate an instantaneous amplitude A(t)′ and an instantaneous phase φ(t)′ as expressed in the following Equations 8 and 9, based on the analytic signal Y(t).

$${A ( t )}^{′} = \vert Y ( t ) \vert = \sqrt{{( {s}_{r} ( t ) )}^{2} + {( H ( {s}_{r} ( t ) ) )}^{2}} \qquad [ Equation 8 ]$$

$${φ ( t )}^{′} = {tan}^{- 1} ( \frac{imag ( Y ( t ) )}{real ( Y ( t ) )} ) = {tan}^{- 1} ( \frac{H ( {s}_{r} ( t ) )}{{s}_{r} ( t )} ) \qquad [ Equation 9 ]$$

An instantaneous frequency f(t)′ may represent a time rate of a phase, and an instantaneous frequency f(t)′ may be calculated based on the following Equation 10.

$${f ( t )}^{′} = \frac{1}{2 π} \frac{d φ ( t )}{dt} \qquad [ Equation 10 ]$$

When it is assumed that a center frequency has a value varying over time instead of f_{c} which is a fixed frequency, the biosignal may be represented by the periodic signal (for example, the second periodic signal) having the instantaneous frequency f(t)′. The first periodic signal S(t) of Equation 1 may be again expressed as the second periodic signal Sₙ(t), based on the instantaneous frequency f(t)′. Here, n may denote the number of times the second signal processing is performed. Aᵥ(t) may be the instantaneous amplitude A(t)′ of Equation 8, and φᵥ(t) may be the instantaneous phase φ(t)′ of Equation 9.

$${S}_{n} ( t ) = {A}_{v} ( t ) cos ( {\int }_{0}^{t} 2 π {f ( τ )}^{′} d τ + {φ}_{v} ( t ) ) + C ( t ) \qquad [ Equation 11 ]$$

The processor may perform the second signal processing based on VFCDM on the second periodic signal Sₙ(t) in operation S122. The second signal processing S122 may include complex demodulation S21, low-pass filtering S22, signal reconstruction S23, Hilbert transform S24, and time-varying frequency calculation S25.

The processor may complex-demodulate the second periodic signal Sₙ(t) in operation S21. The second periodic signal Sp (t) may be multiplied by

$${e}^{- j {\int }_{0}^{t} 2 π f ( τ ) ′ d τ} ,$$

and a result thereof may be expressed as the following Equation 12. The second periodic signal Sₙ(t) may be shifted by −∫₀^{t}2πf(τ)′dτ, and thus, a center frequency thereof may be 0. In other words, the center frequency of the second periodic signal Sₙ(t) may be modulated.

$${S}_{v . n} ( t ) = {S}_{n} ( t ) \cdot {e}^{- j {\int }_{0}^{t} 2 π {f ( τ )}^{′} d τ} = \frac{{A}_{v} ( t )}{2} {e}^{j {φ}_{v} ( t )} + \frac{{A}_{v} ( t )}{2} {e}^{- j ( {\int }_{0}^{t} 4 π f ( τ ) ′ d τ + {φ}_{v} ( t ) )} + C ( t ) {e}^{- j {\int }_{0}^{t} 2 π f ( τ ) ′ d τ} \qquad [ Equation 12 ]$$

The processor may perform low-pass filtering on a complex-demodulated signal S_{v.n}(t) in operation S22. The processor may apply a low-pass filter, having a cut-off frequency which is less than the center frequency −∫₀^{t}2πf(τ)′dτ, to the complex-demodulated signal S_{v.n}(t). Therefore, a filtered signal S_{lp.n}(t) of the following Equation 13 may be obtained.

$${S}_{lp . n} ( t ) = \frac{{A}_{v} ( t )}{2} {e}^{j {φ}_{v} ( t )} \qquad [ Equation 13 ]$$

An amplitude Aᵥ(t) and a phase φᵥ(t) may be respectively expressed as the following Equation 14 and Equation 15.

$${A}_{v} ( t ) = 2 \vert {S}_{lp . n} ( t ) \vert \qquad [ Equation 14 ]$$

$${φ}_{v} ( t ) = {tan}^{- 1} ( \frac{imag ( {S}_{lp . n} ( t ) )}{real ( {S}_{lp . n} ( t ) )} ) \qquad [ Equation 15 ]$$

The processor may reconstruct the second periodic signal (i.e., the biosignal) by using the amplitude Aᵥ(t) and the phase φᵥ(t) in operation S24, and a reconstructed second periodic signal S_{r.n}(t) may be expressed as the following Equation 16.

$${s}_{r . n} ( t ) = {A}_{v} ( t ) cos ( {\int }_{0}^{t} 2 π {f ( τ )}^{′} d τ + {φ}_{v} ( t ) ) \qquad [ Equation 16 ]$$

The processor may apply Hilbert transform to the reconstructed second periodic signal S_{r.n}(t) in operation S24. The processor may calculate a time-varying frequency, based on an analytic signal calculated by Hilbert transform in operation S25. The processor may calculate a time-varying amplitude Aₙ(t)′ and a time-varying phase φₙ(t)′, based on the following Equations 17 and 18.

$${{A}_{n} ( t )}^{′} = \sqrt{{( {{s}_{r . n} ( t )}^{′} )}^{2} + {( H ( {{s}_{r . n} ( t )}^{′} ) )}^{2}} \qquad [ Equation 17 ]$$

$${{φ}_{n} ( t )}^{′} = {tan}^{- 1} ( \frac{H ( {s}_{r . n} ( t ) )}{{s}_{r . n} ( t )} ) \qquad [ Equation 18 ]$$

The processor may calculate a time-varying frequency fₙ(t)′ as expressed in the following Equation 19, based on φₙ(t)′.

$${{f}_{n} ( t )}^{′} = \frac{1}{2 π} \frac{d {φ}_{n} ( t ) ′}{dt} \qquad [ Equation 19 ]$$

A time-frequency spectrum may be generated based on the time-varying amplitude Aₙ(t)′, the time-varying phase φₙ(t)′, and the time-varying frequency fₙ(t)′.

In one or more embodiments, the processor may iteratively perform the second signal processing S122. The processor may second-perform the second signal processing S122 on a periodic signal having the time-varying frequency fₙ(t)′ as a center frequency. For example, the time-varying frequency fₙ(t)′ may be applied to the second periodic signal Sₙ(t) based on Equation 11.

As described above, the processor may perform the second signal processing S122 N (where N may be an integer of 2 or more) times, and a time-varying frequency fₙ(t)′ calculated through n^{th}-performed second signal processing may be applied as a center frequency of a periodic signal on which second signal processing is (n+1)th-performed. Also, a time-varying amplitude Aₙ(t)′ calculated through the n^{th}-performed second signal processing may be applied as an amplitude Aᵥ(t) of the periodic signal on which the second signal processing is (n+1)th-performed, and a time-varying phase φₙ(t)′ calculated through the n^{th}-performed second signal processing may be applied as a phase φᵥ(t) of the periodic signal on which the second signal processing is (n+1)^{th}-performed.

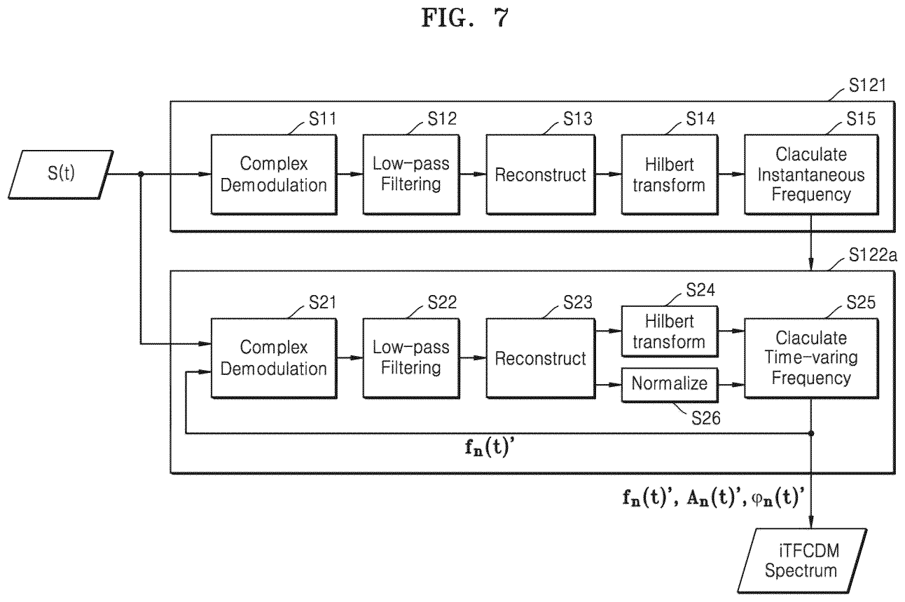

FIG. 7 illustrates signal processing based on iTFCDM according to one or more embodiments. FIG. 7 is a modified embodiment of FIG. 6. Therefore, repeated descriptions are omitted.

Referring to FIG. 7, second signal processing S122a may include complex demodulation S21, low-pass filtering S22, signal reconstruction S23, Hilbert transform S24, normalization S26, and time-varying frequency calculation S25. Comparing with the second signal processing S122 of FIG. 6, the second signal processing S122a of FIG. 7 may further include the normalization S26. A processor may normalize an amplitude by using a maximum value, a minimum value, a middle value, and a standard normal distribution value of the amplitude with respect to a time axis. For example, the processor may divide, by a maximum value A_{v,max}(t), an amplitude Aᵥ(t) calculated based on Equation 14 to normalize. Here, the maximum value A_{v,max}(t) may denote a maximum value within a full frequency range (for example, frequency range from minimum frequency to maximum frequency).

A normalization result value (for example, a normalized value) may be applied as a weight Wₙ of an instantaneous amplitude Aₙ(t)′ in time-varying frequency calculation operation S25. The instantaneous amplitude Aₙ(t)′ may be calculated based on the following Equation 20.

$${{A}_{n} ( t )}^{′} = {W}_{n} \cdot \sqrt{{( {{s}_{r . n} ( t )}^{′} )}^{2} + {( H ( {{s}_{r . n} ( t )}^{′} ) )}^{2}} \qquad [ Equation 20 ]$$

A weight may be applied to an instantaneous amplitude with respect to all center frequencies.

As described above, in signal processing based on iTFCDM, a method where a weight is assigned to an amplitude may be referred to as weighted iTFCDM (wiTFCDM). Accordingly, a signal of a center frequency may be enhanced.

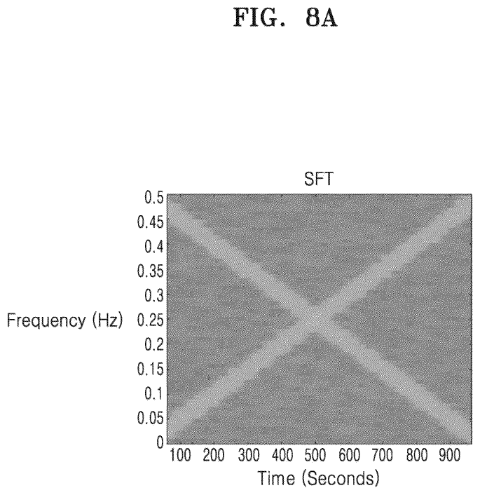

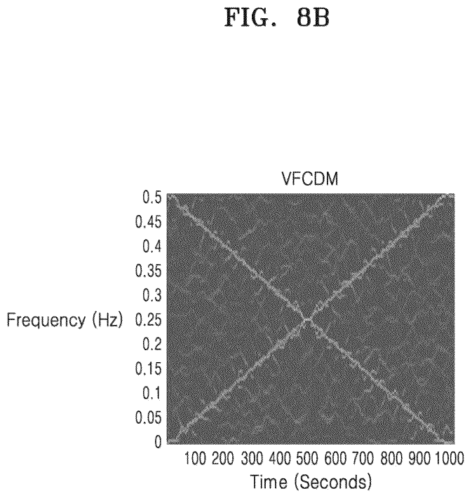

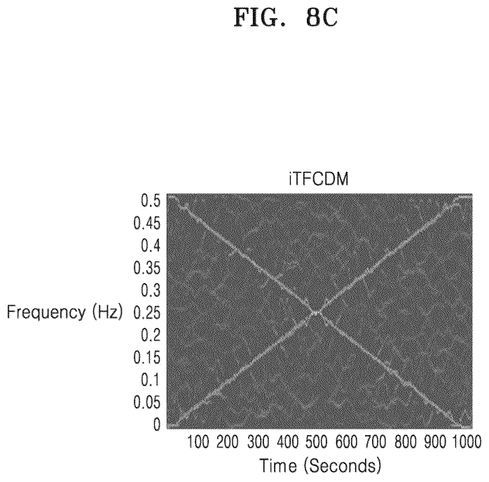

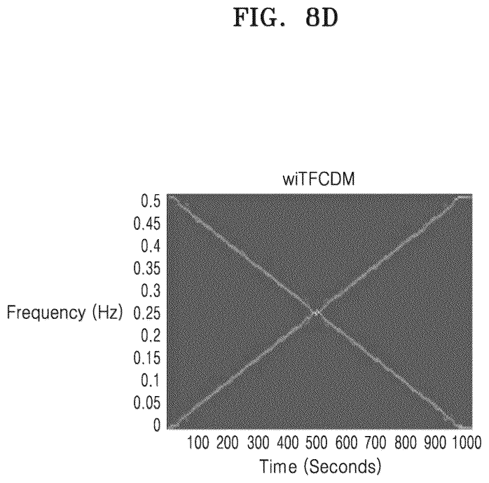

FIGS. 8A to 8D illustrate a time-frequency spectrum of time-series signals.

FIG. 8A shows a spectrum obtained based on short time Fourier transform (STFT), FIG. 8B shows a spectrum obtained based on VFCDM, FIG. 8C shows a spectrum obtained based on iTFCDM according to one or more embodiments, and FIG. 8D shows a spectrum obtained based on wiTFCDM. Each spectrum may represent time-varying analysis of a cross-chirped signal including Gaussian white noise of about 10 dB.

It may be seen that the spectrum obtained based on STFT in FIG. 8A is relatively low in resolution. STFT may have a trade-off relationship between a time resolution and a frequency resolution. A frequency resolution may decrease when a time resolution increases, but when a frequency resolution increases, a time resolution may decrease. Therefore, time-frequency analysis of a high resolution may be difficult.

A time-frequency spectrum of a high resolution may be obtained based on VFCDM in FIG. 8B. It may be seen that a cross-chirped signal appears clearly.

It may be seen that a cross-chirped signal is more clearly shown in a spectrum obtained based on iTFCDM in FIG. 8C and a spectrum obtained based on wiTFCDM in FIG. 8D than the spectrum of FIG. 8B. Also, it may be seen that a noise component is clearly removed in the spectrum of FIG. 8D.

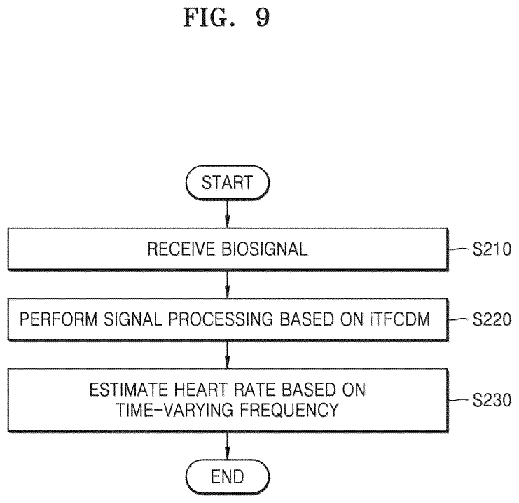

FIG. 9 is a flowchart illustrating a signal processing method according to one or more embodiments. The signal processing method of FIG. 9 may be performed by at least one processor (for example, the processor 110 of FIG. 1).

Referring to FIG. 9, the processor may receive a biosignal from a sensor in operation S210. The biosignal may be an ECG signal and/or a PPG signal.

The processor may perform signal processing based on iTFCDM on the biosignal in operation S220. A time-varying frequency including signal processing may be calculated. The processor may perform signal processing based on wiTFCDM. Hereinafter, in the disclosure, iTFCDM may have a meaning which includes wiTFCDM.

The processor may estimate a heart rate, based on a time-varying frequency. For example, the processor may perform signal processing based on iTFCDM on an ECG signal or a PPG signal and may estimate, as a heart rate, a time-varying frequency calculated as a result of the signal processing.

In one or more embodiments, the processor may obtain a time-frequency spectrum based on iTFCDM. As the signal processing based on iTFCDM is performed on the ECG signal or the PPG signal, a signal may be amplified, and noise may decrease.

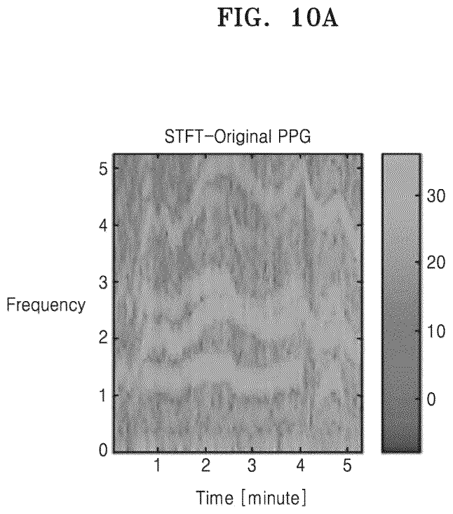

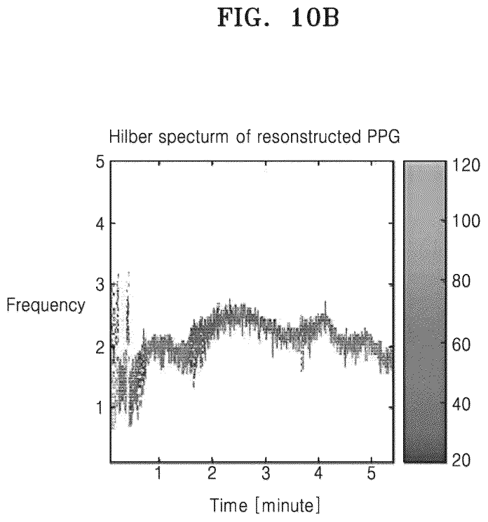

FIGS. 10A and 10B illustrate a time-frequency spectrum of a PPG signal.

FIG. 10A shows a spectrum obtained based on STFT, and FIG. 10B shows a spectrum obtained based on VFCDM.

In FIG. 10A, a signal may strongly appear in various frequencies. Therefore, it may not be easy to extract a center frequency for estimating a heart rate. However, in FIG. 10B, a center frequency may clearly appear, and thus, the extraction of a center frequency may be easy. In iTFCDM according to one or more embodiments, as VFCDM is iteratively performed, a center frequency may be more clearly shown. An extracted center frequency may be estimated as a heart rate.

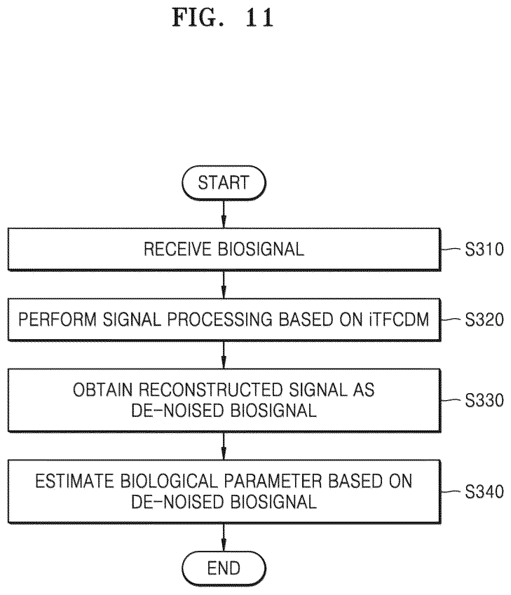

FIG. 11 is a flowchart illustrating a signal processing method according to one or more embodiments. The signal processing method of FIG. 11 may be performed by at least one processor (for example, the processor 110 of FIG. 1).

Referring to FIG. 11, the processor may receive a biosignal from a sensor in operation S310. The biosignal may be an ECG signal and/or a PPG signal.

The processor may perform signal processing based on iTFCDM on the biosignal in operation S320. In a process where first signal processing and one or more second signal processing are performed, an instantaneous frequency, a time-varying frequency, a time-varying amplitude, a time-varying phase, an instantaneously reconstructed first periodic signal, and one or more reconstructed second periodic signals may be calculated as processing signals.

The processor may obtain, as a de-noised signal, a reconstructed signal (for example, the reconstructed first periodic signal or the reconstructed second periodic signal) calculated through at least one of the first signal processing and the one or more second signal processing in operation S330. As described above with reference to FIG. 6, in a reconstructed signal, a signal component may be amplified, and noise may decrease.

The processor may estimate bio-information, based on a de-noised biosignal in operation S340. For example, the processor may estimate a biological parameter such as a heart rate, a breathing rate, oxygen saturation, blood pressure, heart rate variability, stress, or sleep apnea, based on the de-noised biosignal.

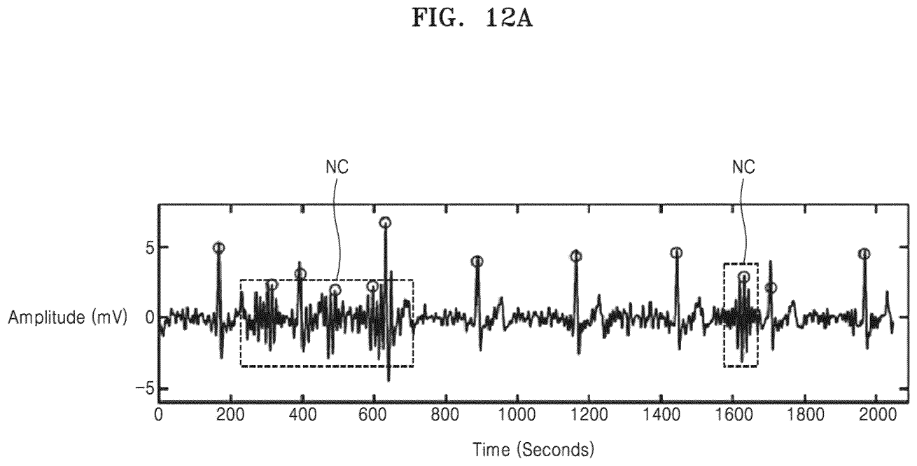

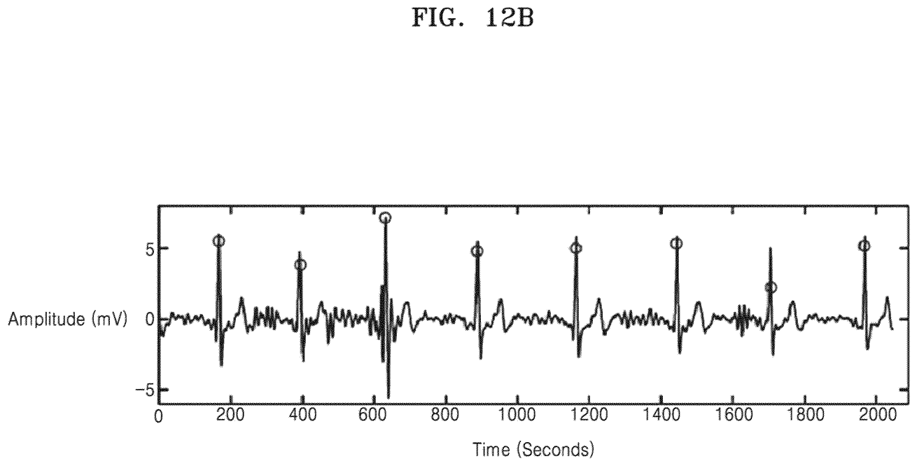

FIG. 12A illustrates an ECG signal, and FIG. 12B illustrates a reconstructed ECG signal generated in a signal processing operation according to one or more embodiments.

Referring to FIG. 12A, an ECG signal may be distorted because a noise component NC is added to the ECG signal. In addition to peaks by heartbeat, peaks by the noise component NC may be added to the ECG signal. As described above with reference to FIG. 4A, a heart rate may be estimated based on an RR interval. However, an inaccurate heart rate may be estimated by the peaks by the noise component NC.

FIG. 12B illustrates a reconstructed ECG signal generated in a signal processing operation based on iTFCDM according to one or more embodiments. A noise component may be removed in the reconstructed ECG signal, and thus, peaks by the noise component may be removed. Accordingly, an accurate heart rate may be estimated based on the reconstructed ECG signal.

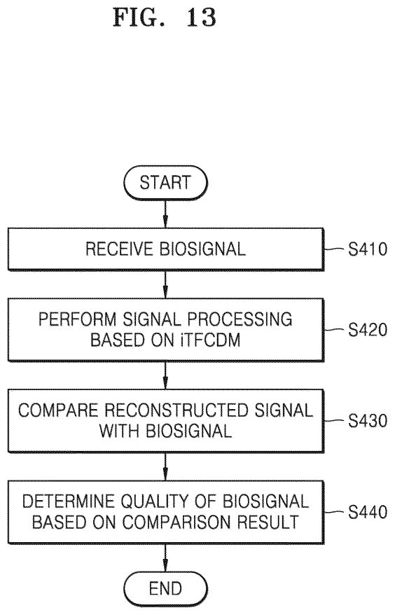

FIG. 13 is a flowchart illustrating a signal processing method according to one or more embodiments. The signal processing method of FIG. 13 may be performed by at least one processor (for example, the processor 110 of FIG. 1.

Referring to FIG. 13, the processor may receive a biosignal from a sensor in operation S410 and may perform signal processing based on iTFCDM on the biosignal in operation S420.

The processor may compare the biosignal with a reconstructed signal calculated through at least one of first signal processing and/or one or more second signal processing in operation S430. The processor may determine the quality of the biosignal, based on a comparison result in operation S440. For example, the processor may calculate a similarity between the biosignal and the reconstructed signal and may determine a quality index, based on the similarity. The quality index may represent the quality of an original biosignal. As a similarity between the original biosignal and the reconstructed signal increases, the quality of the biosignal may be determined to be high.

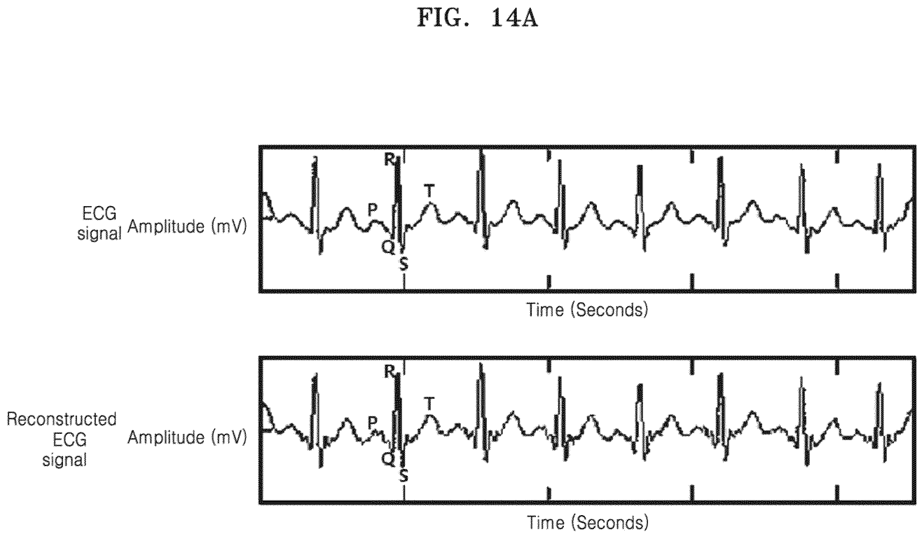

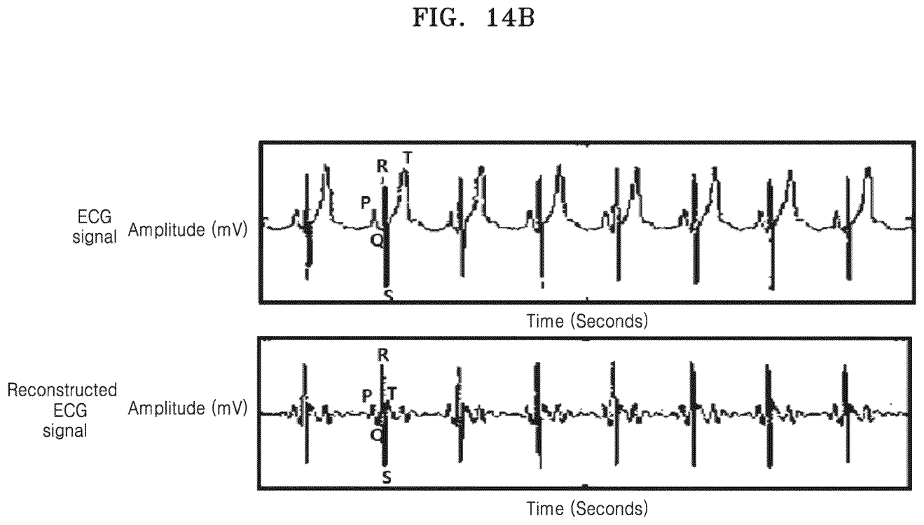

FIG. 14A illustrates a case where a similarity between an ECG signal and a reconstructed ECG signal is high. As illustrated, feature points (for example, PQRST waves) in an original ECG signal may have a normal form. The reconstructed ECG signal may be a signal where noise is removed and a signal component is amplified in the original ECG signal. Accordingly, as the quality of the original ECG signal increases, a similarity between the ECG signal and the reconstructed ECG signal may be high.

FIG. 14B illustrates a case where a similarity between an ECG signal and a reconstructed ECG signal is low. Feature points in an original ECG signal may have an abnormal form. T wave may very high, and S wave may be very deep. Such abnormal feature points may be removed as noise in the reconstructed ECG signal. For example, T wave and S wave in the reconstructed ECG signal may differ from T wave and S wave in the original ECG signal. Accordingly, a similarity between the reconstructed ECG signal and the original ECG signal may be low.

Referring again to FIG. 13, the processor may determine quality or a quality index, based on a reference value. For example, when the similarity is less than a first reference value, the processor may determine that the quality of an original biosignal is low, and when the similarity is greater than or equal to the first reference value, the processor may determine that the quality of an original biosignal is high. However, the disclosure is not limited thereto, and the processor may determine a quality index having various levels (or range), based on the similarity.

In one or more embodiments, the processor may estimate bio-information, based on the quality index. For example, when a quality index of a biosignal is low (for example, the quality index may be less than a threshold value), the processor may exclude the use of the biosignal in a bio-information estimation operation. As another example, the processor may apply a weight to a biosignal in a bio-information estimation operation, based on a quality index of each biosignal.

In one or more embodiments, the processor may determine the reliability of estimated bio-information, based on a quality index. For example, when a quality index of a biosignal used in an operation of estimating estimated bio-information is low, the processor may determine that the reliability of the estimated bio-information is low. As another example, the processor may use a quality index as a weight when determining the reliability of estimated bio-information.

As described above with reference to FIGS. 9 to 14B, bio-information may be estimated from a biosignal, based on high resolution time-frequency analysis based on iTFCDM according to one or more embodiments, and the accuracy of bio-information may be enhanced.

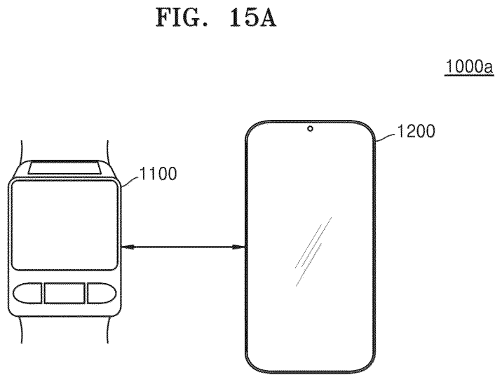

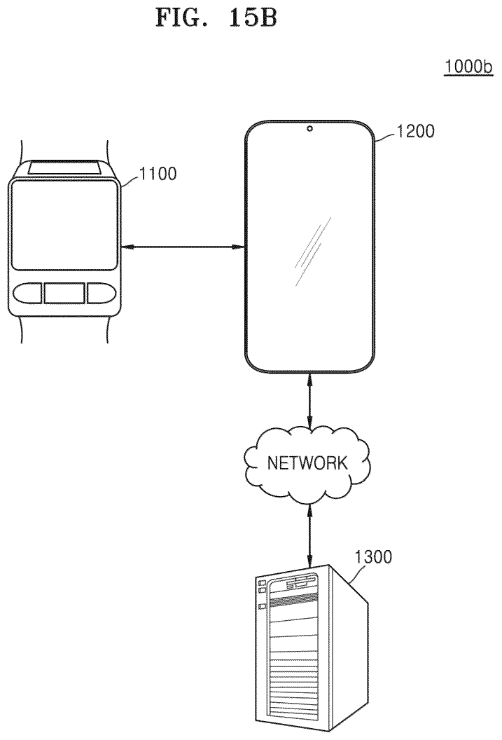

FIG. 15A illustrates a biosignal monitoring system 1000a according to one or more embodiments, and FIG. 15B illustrates a biosignal monitoring system 1000b according to one or more embodiments.

Referring to FIGS. 15A and 15B, each of the biosignal monitoring system 1000a and the biosignal monitoring system 1000b may include a biosignal monitoring device 1100 and a data reception device 1200. The biosignal monitoring system 1000b may further include a server 1300.

The electronic device 100 (for example, a wearable device) described above with reference to FIG. 1 may be applied to the biosignal monitoring device 1100. The above descriptions of the electronic device 100 and an operation thereof may be applied to the present embodiment. The data reception device 1200, for example, may be an electronic device, including a mobile communication interface, such as a smartphone, a tablet personal computer (PC), or a mobile communication device. The server 1300 may be, for example, a medical server or a cloud server.

The biosignal monitoring device 1100 may measure a biosignal such as a PPG signal, an ECG signal, or an EMG signal and may perform ultra-high resolution time-frequency analysis through signal processing based on iTFCDM described above on the biosignal. Also, a level of a biosignal may be enhanced in a signal processing operation, and a reconstructed biosignal where noise is reduced may be generated. The biosignal monitoring device 1100 may estimate bio-information, based on at least one processing signal generated as a signal processing result or a signal processing operation. Accordingly, the accuracy of bio-information may be enhanced.

The biosignal monitoring device 1100 may output the estimated bio-information (for example, a heart rate, a breathing rate, oxygen saturation, blood pressure, heart rate variability, stress, or sleep apnea) through a display or a speaker. In one or more embodiments, the biosignal monitoring device 1100 may diagnose disease, based on the estimated bio-information. For example, the biosignal monitoring device 1100 may determine atrial fibrillation, based on a heart rate, and may provide a user with event information indicating atrial fibrillation detection.

The data reception device 1200 may transmit the biosignal, the estimated bio-information, or the diagnosed disease to the server 1300. In one or more embodiments, the biosignal monitoring device 1100 may include a mobile communication interface and may directly transmit the biosignal, the estimated bio-information, or the diagnosed disease to the server 1300. A medical team may detect (diagnose) atrial fibrillation, based on an ECG signal transmitted to the server 1300.

In one or more embodiments, the data reception device 1200 or the server 1300 may perform ultra-high resolution time-frequency analysis through signal processing based on iTFCDM described above on the biosignal received from the biosignal monitoring device 1100.

In the biosignal monitoring system 1000a and the biosignal monitoring system 1000b according to one or more embodiments, the biosignal monitoring device 1100, the data reception device 1200, or the server 1300 may perform signal processing based on iTFCDM on the biosignal measured by the biosignal monitoring device 1100 to analyze ultra-high resolution time-frequency. Also, bio-information may be estimated based on an analysis result, and thus, the accuracy of bio-information may be enhanced.

Hereinabove, exemplary embodiments have been described in the drawings and the specification. Embodiments have been described by using the terms described herein, but this has been merely used for describing the disclosure and has not been used for limiting a meaning or limiting the scope of the disclosure defined in the following claims. Therefore, it may be understood by those of ordinary skill in the art that various modifications and other equivalent embodiments may be implemented from the disclosure. Accordingly, the spirit and scope of the disclosure may be defined based on the spirit and scope of the following claims.

While the disclosure has been particularly shown and described with reference to embodiments thereof, it will be understood that various changes in form and details may be made therein without departing from the spirit and scope of the following claims.

1. A biosignal processing method comprising:
receiving a biosignal from a sensor;
performing first signal processing on the biosignal, based on variable frequency complex demodulation (VFCDM);
performing, at least once, second signal processing based on the VFCDM on a periodic signal having an instantaneous frequency as a center frequency, the instantaneous frequency being obtained based on the first signal processing;
estimating bio-information, based on at least one processing signal obtained based on the second signal processing; and
displaying the bio-information.

2. The biosignal processing method of claim 1,
wherein the second signal processing is performed N times, where N is an integer of at least 2, and
wherein (n+1)^{th} second signal processing is performed on the periodic signal having a time-varying frequency as the center frequency, the time-varying frequency being obtained based on n^{th}-performed second signal processing, where n is an integer which is at least 1 and less than N.

3. The biosignal processing method of claim 1, wherein the first signal processing comprises:
complex-demodulating the biosignal to generate a first signal;
removing a signal component, which is greater than or equal to the center frequency, from the first signal to generate a second signal;
reconstructing the biosignal, based on an amplitude and a phase, each obtained from the second signal; and
calculating the instantaneous frequency from a reconstructed biosignal.

4. The biosignal processing method of claim 1, wherein the second signal processing comprises:
complex-demodulating the periodic signal to generate a third signal;
removing a signal component, which is greater than or equal to the center frequency, from the third signal to generate a fourth signal;
reconstructing the periodic signal, based on an amplitude and a phase, each obtained from the fourth signal; and
obtaining a time-varying frequency from a reconstructed periodic signal.

5. The biosignal processing method of claim 4, wherein the obtaining of the time-varying frequency comprises:
performing Hilbert transform on the reconstructed periodic signal to obtain a time-varying amplitude and a time-varying phase of the reconstructed periodic signal; and
calculating the time-varying frequency, based on the time-varying phase.

6. The biosignal processing method of claim 5,
wherein the second signal processing further comprises normalizing the amplitude obtained from the fourth signal to obtain a weight, and
wherein the weight is applied to the time-varying amplitude of the reconstructed periodic signal.

7. The biosignal processing method of claim 4, wherein the estimating of the bio-information comprises:
obtaining the reconstructed periodic signal as a de-noised biosignal; and
estimating a biological parameter, based on the de-noised biosignal.

8. The biosignal processing method of claim 4, wherein the estimating of the bio-information comprises:
comparing the reconstructed periodic signal with the biosignal; and
determining quality of the biosignal, based on a comparison result.

9. The biosignal processing method of claim 1,
wherein the biosignal comprises at least one of an electrocardiogram (ECG) signal and a photoplethysmogram (PPG) signal, and
wherein the estimating of the bio-information comprises estimating a heart rate, based on a time-varying frequency obtained as a result of the second signal processing.

10. The biosignal processing method of claim 1, wherein the biosignal has periodicity based on time.

11. A biosignal processing method comprising:
performing first signal processing based on variable frequency complex demodulation (VFCDM) on a first periodic signal obtained from a biosignal;
performing second signal processing based on the VFCDM on a second periodic signal having an instantaneous frequency as a second center frequency, the instantaneous frequency being obtained based on the first signal processing;
performing third signal processing based on the VFCDM on a third periodic signal having a time-varying frequency as a third center frequency, the time-varying frequency being obtained based on the second signal processing;
estimating bio-information, based on at least one processing signal obtained based on the first signal processing, the second signal processing, and the third signal processing; and
displaying the bio-information.

12. The biosignal processing method of claim 11, wherein each of the second signal processing and the third signal processing comprises:
complex-demodulating a periodic signal that is input;
low-pass filtering a complex-demodulated signal by using a low-pass filter having a cut-off frequency which is lower than a center frequency;
reconstructing the periodic signal, based on an amplitude and a phase, each obtained from a filtered signal; and
obtaining the time-varying frequency from a reconstructed periodic signal.

13. The biosignal processing method of claim 12, wherein the obtaining of the time-varying frequency comprises:
performing Hilbert transform on the reconstructed periodic signal; and
calculating a time-varying amplitude, a time-varying phase, and the time-varying frequency, based on an analytic signal generated through the Hilbert transform.

14. The biosignal processing method of claim 13,
wherein each of the second signal processing and the third signal processing comprises normalizing the amplitude obtained from the filtered signal to obtain a weight, and
wherein the weight is applied to the time-varying amplitude of the reconstructed periodic signal.

15. (canceled)

16. (canceled)

17. A biosignal processing apparatus for processing a biosignal, the biosignal processing apparatus comprising:
a display;
a memory storing instructions; and
at least one processor operatively connected to the display and the memory, the at least one processor being configured to execute the instructions to:
perform first signal processing based on variable frequency complex demodulation (VFCDM) on a first periodic signal obtained from the biosignal;
perform, at least once, second signal processing based on the VFCDM on a second periodic signal having an instantaneous frequency as a center frequency, the instantaneous frequency being obtained based on the first signal processing;
estimate bio-information, based on at least one processing signal obtained based on the second signal processing; and
instruct the display to display the bio-information.

18. The biosignal processing apparatus of claim 17, wherein the at least one processor is further configured to execute the instructions to:
complex-demodulate the second periodic signal;
low-pass filter a complex-demodulated signal, based on a cut-off frequency which is lower than the center frequency;
reconstruct the second periodic signal, based on an amplitude and a phase each obtained from a filtered signal; and
obtain a time-varying frequency from a reconstructed second periodic signal.

19. The biosignal processing apparatus of claim 18, wherein the at least one processor is further configured to execute the instructions to:
normalize the amplitude obtained from the filtered signal to obtain a weight; and
apply the weight to an amplitude obtained through Hilbert transform from the reconstructed second periodic signal.

20. The biosignal processing apparatus of claim 18, wherein the at least one processor is further configured to execute the instructions to estimate the bio-information, based on the reconstructed second periodic signal.

21. The biosignal processing apparatus of claim 18, wherein the at least one processor is further configured to execute the instructions to:
compare the reconstructed second periodic signal with the biosignal; and
determine quality of the biosignal, based on a comparison result.

22. The biosignal processing apparatus of claim 17, wherein the at least one processor is further configured to execute the instructions to estimate a heart rate, based on a result of the second signal processing.

23. (canceled)

24. (canceled)

25. (canceled)
